(19) United States
(12) Patent Application Publication
(10) Pub. No.: US 2015/0148755 A1
(43) Pub. Date: May 28, 2015

(54) SYRINGE

(71) Applicant: YUE PFONG INTERNATIONAL TECHNOLOGY CORP., Taichung (TW)

(72) Inventor: YU-LIN LAI, Taichung (TW)

(73) Assignee: YUE PFONG INTERNATIONAL TECHNOLOGY CORP., Taichung (TW)

(21) Appl. No.: 14/091,077

(22) Filed: Nov. 26, 2013

(51) Int. Cl. A61M 5/34

(52) U.S. Cl. CPC A61M 5/344
USPC 604/241

(57) ABSTRACT

A syringe is provided. A front end of a barrel has an annular blocking portion which has at least two projections and at least two engaging slots defined by the projections, and one side of each projection has a guiding face. A connector which has at least two blocking members arranged circumferentially is assembled to the front end of the barrel, and one side of each blocking member has an incline. The blocking members are respectively received in the engaging slots and blocked by the projections. A plunger is movably received in the barrel, wherein a front end thereof is movable and abuttable correspondingly against the bottom of the connector, and the front end of the plunger is connected with the bottom of the connector.

### BACKGROUND OF THE INVENTION

1. Field of the Invention

The present invention relates to a syringe, and more particularly to a syringe whose connector and needle assembly are effectively positionable.

2. Description of the Prior Art

In many areas of the world, a lot of medical personnel and personnel to handle medical waste might be hurt by needles due to inadequate handling of syringes, and therefore might be infected with diseases such as AIDS, hepatitis B, pulmonary tuberculosis (TB) et al. According to statistical data, unsafe injection can cause 1.3 millions of deaths per year all over the world, resulting in hundres billion of medical expenses. Unsafe injection is meant that unsafe injection acts that are applied to patients or carried out by medical personnel or carried out in risky public environment, for example, reuse of throw-away type syringes, defects of aseptic operation such as needle sharing behaviors and improper handling of medical waste et al. To avoid those mentioned above, various types of safety syringes are available in the market. In a safety syringe, the needle assembly is installed to the barrel and can be withdrawn back into the barrel after used. Accordingly, the inventor has researched for year to provide a syringe which is throw-away type and sanitary, and which is designed in a structure that is constructed of few elements.

The present invention is, therefore, arisen to obviate or at least mitigate the above mentioned disadvantages.

### SUMMARY OF THE INVENTION

An object of the present invention is to provide a syringe which is designed in a structure that is constructed of few elements and, however, can ensure entire withdraw of a needle assembly back into the interior of the syringe so as to avoid resue of the syringe.

Another object of the present invention is to provide a syringe whose connector and barrel have cooperatively positionable structures, which can effectively restrict rotation of the connector, so as to avoid disengagement of the connector from the barrel due to improper operation of the needle assembly.

To achieve the above and other objects, a syringe is provided. The syringe includes a barrel, a connector and a plunger. A front end of the barrel has an annular blocking portion, wherein the annular blocking portion has at least two projections and at least two engaging slots defined by the projections, and one side of each projection formed with a guiding face. The connector is assembled to the front end of the barrel, wherein the connector has at least two blocking members arranged circumferentially, and one side of each blocking member is formed with an incline. The blocking members are respectively received in the engaging slots and blocked by the projections so as to restrict rotation of the connector. The plunger is movably received in the barrel, wherein a front end thereof is movable and abuttable correspondingly against the bottom of the connector, and the front end of the plunger is connected with the bottom of the connector. When the connector is assembled into the front end of the barrel, the blocking members and the projections are guided to be blockably positioned through contact of the guiding faces and the inclines.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective exploded view according to an embodiment of the present invention;

FIG. 2 is a cross-sectional view according to an embodiment of the present invention;

FIG. 3 is a partial cross-sectional view according to an embodiment of the present invention;

FIG. 4 is a perspective view showing a connector according to an embodiment of the present invention;

FIG. 5 is a perspective view showing a barrel according to an embodiment of the present invention;

FIG. 6 is another partial cross-sectional view according to an embodiment of the present invention;

FIG. 6A is a partial enlarged drawing of FIG. 6;

FIGS. 7A and 7B are cross-sectional views of FIG. 6 taken along line 7-7;

FIGS. 8 and 9 are views showing a syringe in use according to an embodiment of the present invention;

### DETAILED DESCRIPTION OF THE PREFERRED EMBODIMENTS

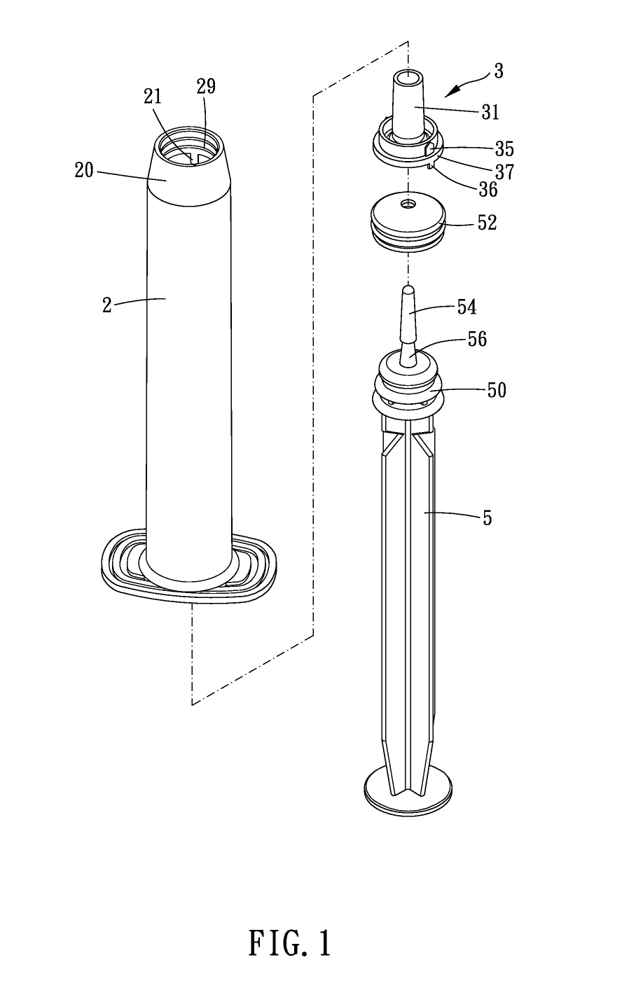

FIGS. 1-7B show a syringe according to an embodiment of the present invention. The syringe includes a barrel 2, a connector 3 and a plunger 5.

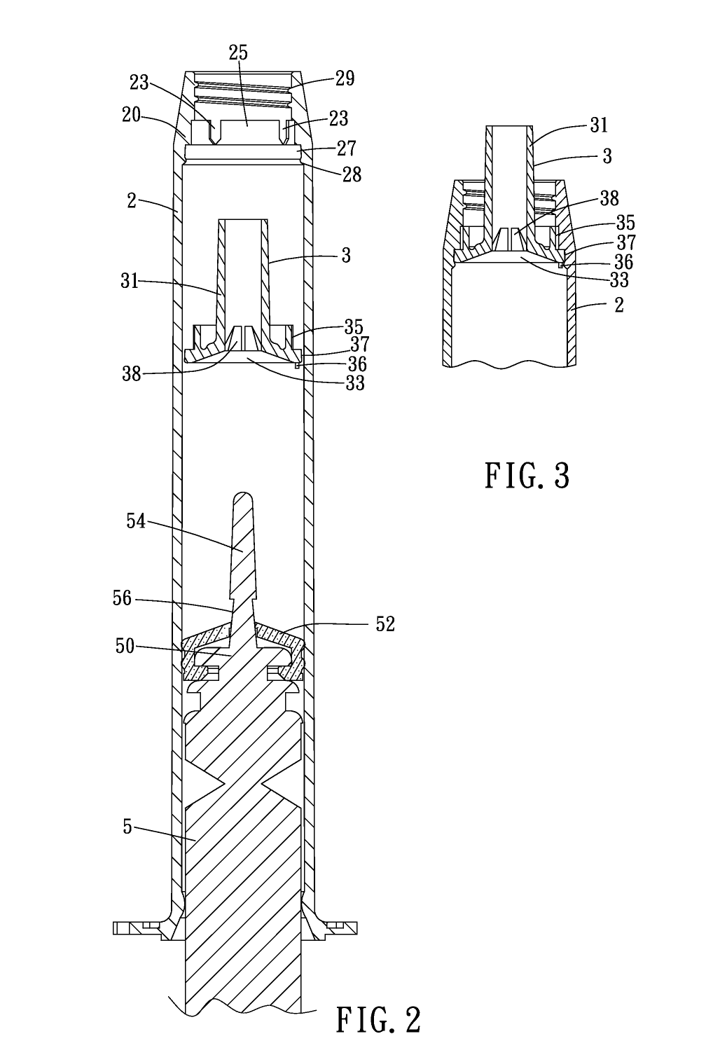

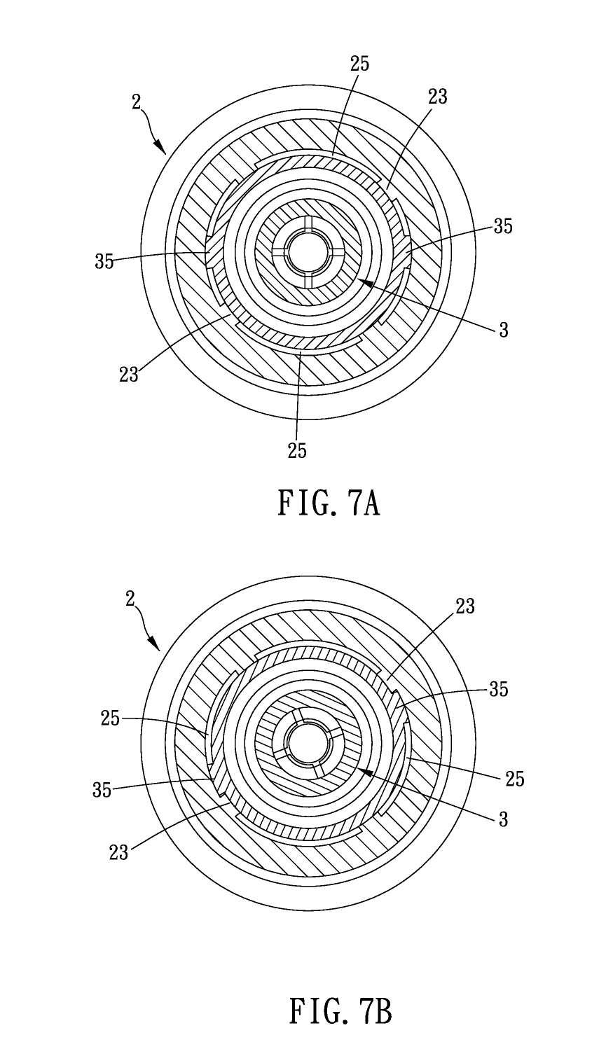

As shown in FIGS. 1, 2, 4 and 5, a front end 20 of an interior of the barrel 2 is formed with an annular blocking portion 21, wherein the annular blocking portion 21 has at least two projections 23 and at least two engaging slots 25. The engaging slots 25 are defined by the projections 23. One side of each projection 23 is formed with a guiding face 230. In this embodiment, each guiding face 230 is V-shaped. Referring further to FIGS. 7A and 7B, in this embodiment, the annular blocking portion 21 has four projections 23 and four engaging slots 25. Each of the four engaging slots 25 is defined by every two neighboring projections 23, so that the front end 20 of the barrel 2 can provide effective restriction to the connector 3. Additionally, the front end 20 of the barrel 2 further has a conical annular groove 27. The conical annular groove 27 is formed under the engaging slot 25, and the conical annular groove 27 and the engaging slot 25 are configured to be stepped, as shown in FIG. 2. A diameter of the conical annular groove 27 is greater than that of the engaging slot 25, for facilitating the positioning of connector 3.

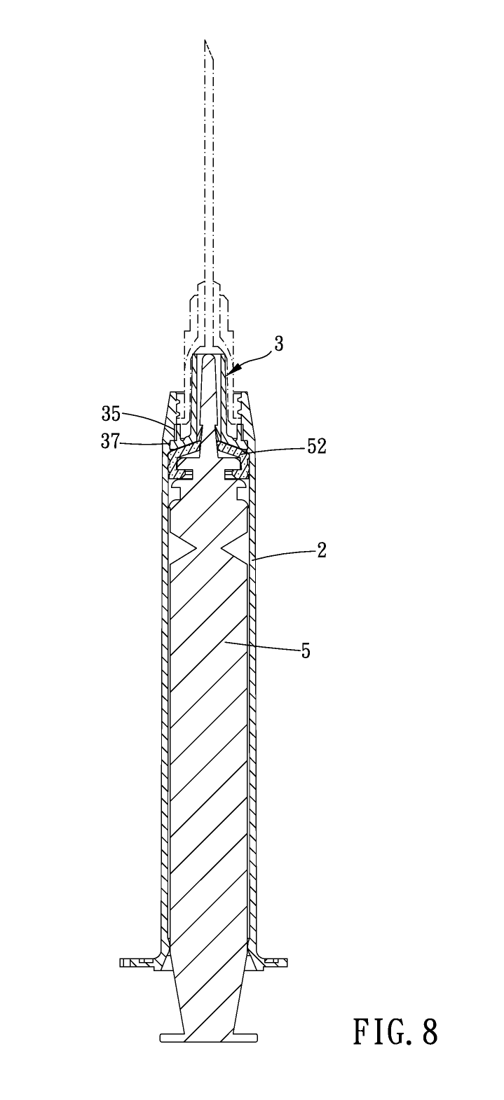

The front end 20 of the barrel 2 has a protrusion 28 protruding from a lower edge of the conical annular groove 27. The bottom of the connector 3 is blocked by the protrusion 28, so as to firmly block the connector 3 at the front end 20 of the barrel 2. The front end 20 of the barrel 2 is formed with a threaded portion 29 for threading connection of a needle assembly (as shown in FIG. 8).

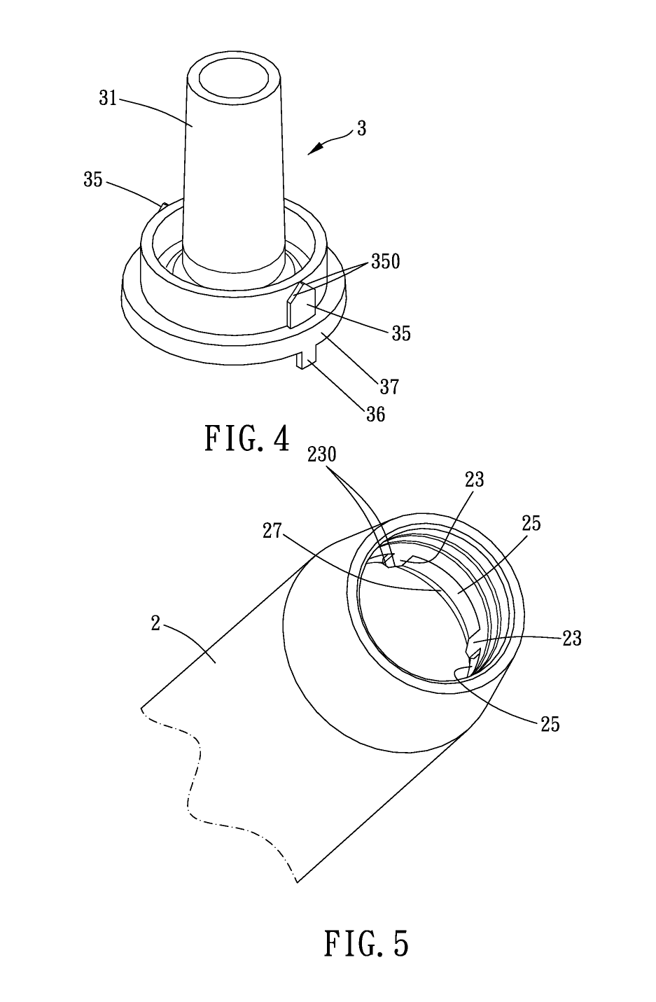

As shown in FIGS. 4, 7A and 7B, the connector 3 is installed in the front end 20 of the barrel 2, and has two blocking members 35 arranged circumferentially. One side of each blocking member 35 has an incline 350. In this embodiment, each incline 350 is V-shaped. As the connector 3 is moved within the barrel 2 and located at the front end 20, the blocking members 35 are respectively received in the engaging slots 25 and blocked by the projections 23, so as to restrict rotation of the connector 3 in the barrel 2. The connector 3 extends upwardly to form an extending member 31 for connection of the needle assembly (as shown in FIG. 8). The connector 3 further has a conical annular member 37 arranged circumferentially, wherein the conical annular member 37 is formed under the blocking member 35, and the conical annular member 37 and the blocking member 35 are configured to be stepped, so that when the connector 3 is located into the front end 20 of the barrel 2, the conical annular member 37 is correspondingly engageable with the conical annular groove 27.

As the connector 3 is assembled into the front end 20 of the barrel 2, the blocking members 35 and the projections 23 are guided to be blockably positioned through contact of every corresponding guiding face 230 and incline 350. As a result, the blocking member 35 can be engaged correspondingly with the engaging slot 25 and blocked by the projection 23, so as to blockably position the blocking member 35 and the projection 23 and avoid oppositely impact of the blocking member 35 and the projection 23 and damage.

As shown in FIGS. 2, 3 and 6, an outer diameter of the conical annular member 37 is greater than that of the blocking member 35. As the connector 3 is assembled in the barrel 2, the conical annular member 37 is in close fit with the conical annular groove 27 and the blocking member 35 is blocked correspondingly within the engaging slot 25. Therefore, not only rotation of the connector 3 relative to the barrel 2 can be restricted, but also the connector 3 and the barrel 2 can be firmly cooperatively positioned without additional element such as O-ring or seal and effectively prevent leakage of the injection liquid in the syring. Besides, the interior of the connector 3 integrally extends to form a restraining portion 38. The restraining portion 38 extends inwardly and upwardly to form a tapered structure, the bottom of the connector 3 is formed with a recessed portion 33, and the bottom of the connector 3 has an abutting member 36 projecting therefrom.

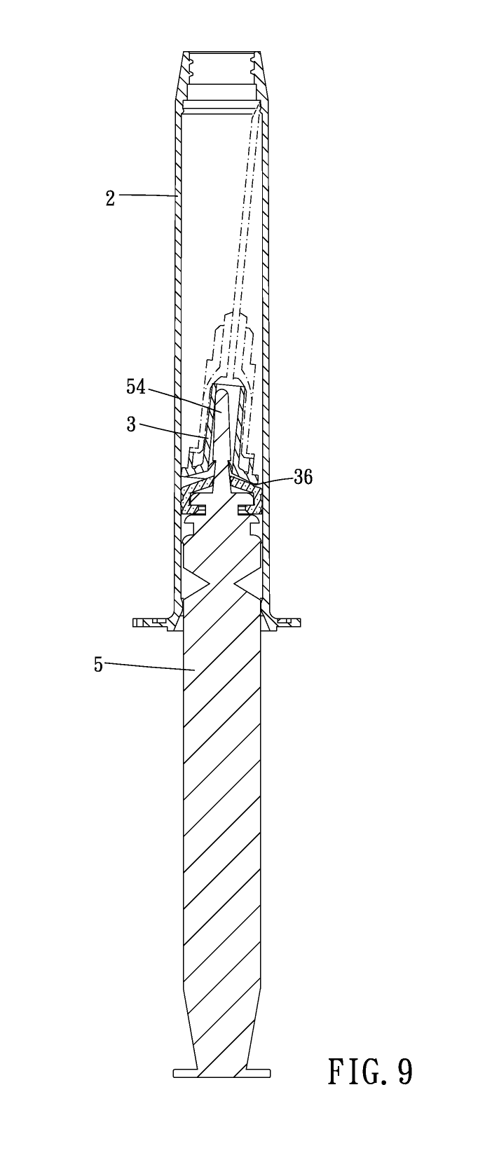

The plunger 5 is movably received in the barrel 2, the front end 50 of the plunger 5 is movable and abuttable correspondingly against and connected with the bottom of the connector 3. Specifically, the front end 50 of the plunger 5 has a piston 52 and a rod member 54 which is protrusive outside the piston 52. The piston 52 is movable and abuttable correspondingly against the bottom of the connector 3, and the piston 52 is fitted to the recessed portion 33 of the connector 3. Whereby, the injection liquid in the syringe can be effectively delivered into the extending member 31 of the connector 3, and it ensures that the injection liquid can be entirely delivered into the needle assembly (as shown in FIG. 8); besides, the rod member 54 can be correspondingly received in the connector 3, facilitating restriction of the rod member 54 to the tapered structure of the restraining portion 38. The rod member 54 is formed with a restraining slot 56. As the rod member 54 is received in the connector 3, the tapered structure of the restraining portion 38 can be blocked within the restraining slot 56 of the rod member 54 and partial surface of the piston 52 is simultaneously shoved by the abutting member 36 of the connector 3 (as shown in FIG. 9).

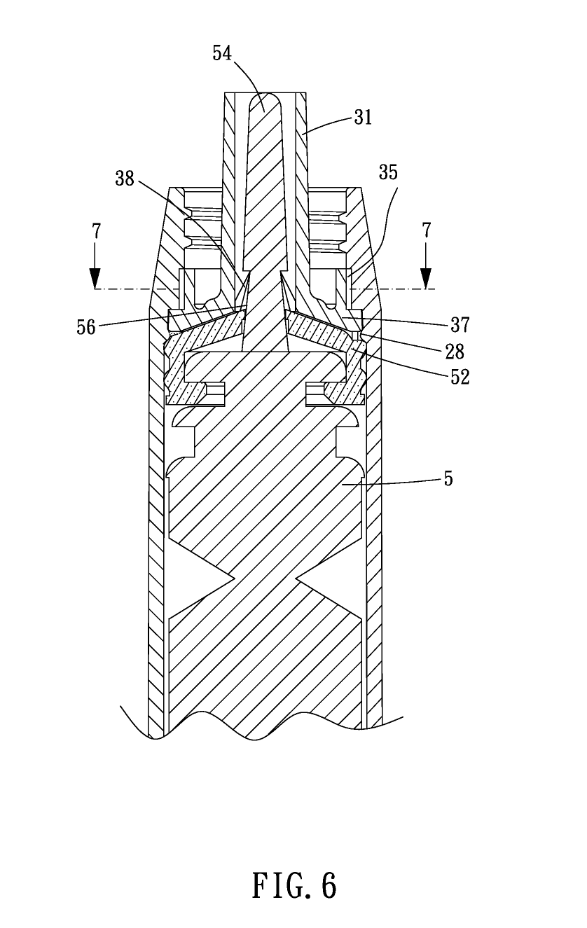

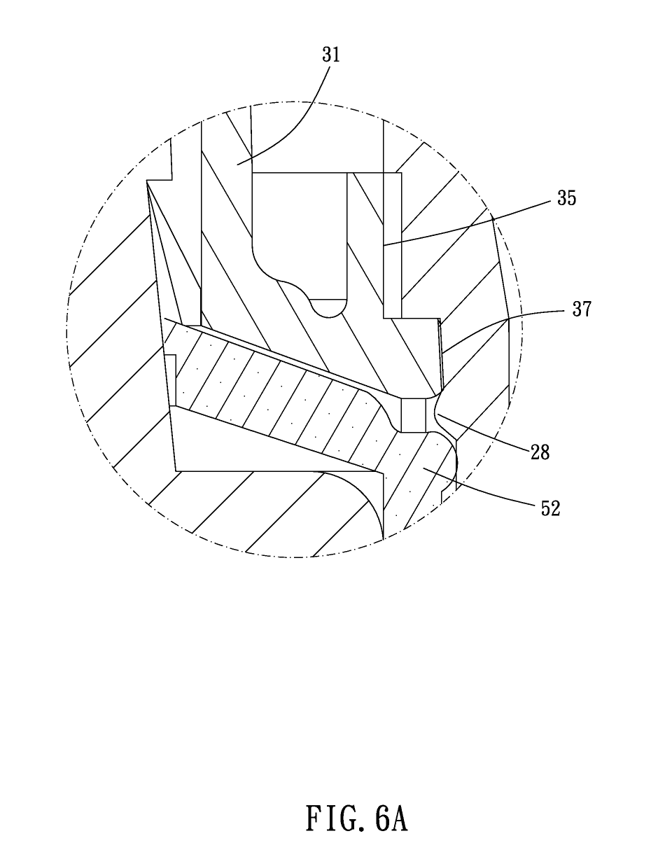

As shown in FIGS. 6, 8 and 9, in use, the plunger 5 is pushed forward to move the front end 50 toward the front end 20 of the barrel 2, so as to fit the piston 52 of the plunger 5 into the recessed portion 33 of the connector 3, which can effectively and entirely discharge the injection liquid out from the barrel 2, at the same time, the rod member 54 is received in the connector 3, so that the restraining portion 38 of the connector 3 can be effectively blocked by the restraining slot 56 of the rod member 54. Thereafter, the plunger 5 is pulled back and the connector 3 and the needle assembly (as shown in FIG. 9) are certainly pulled back into the barrel 2. Additionally, since the abutting member 36 of the connector 3 can shove the piston 52 of the plunger 5, the connector 3 and the needle assembly are urged to lean (as shown in FIG. 9) so that the connector and the needle assembly can be certainly received and blocked in the barrel, and thus it is safe and sanitary, and can avoid resue of the syringe.

In sum, through contact of the guiding faces and the inclines, the blocking members and the projections are guided to be blockably positioned quickly, and it can avoid oppositely impact of the blocking member and the projection and damage, so as to avoid disengagement of the connector from the barrel due to improper operation of the needle assembly. Furthermore, it can ensure entire withdraw of a needle assembly back into the interior of the syringe, and thus it is safe and sanitary. Moreover, the syringe of the invention not only has few emenmets but also can ensure that the connector and the needle assembly be certainly withdrawn back and blocked in the barrel 2, avoiding resue of the syringe.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A syringe, including:
a barrel, a front end of an interior thereof formed with an annular blocking portion, the annular blocking portion having at least two projections and at least two engaging slots, the engaging slots defined by the projections, one side of each projection formed with a guiding face;
a connector, assembled to the front end of the barrel, the connector having at least two blocking members arranged circumferentially, one side of each blocking member formed with an incline, the blocking members respectively received in the engaging slots and blocked by the projections so as to restrict rotation of the connector; and
a plunger, movably received in the barrel, a front end thereof being movable and abuttable correspondingly against the bottom of the connector, and the front end of the plunger being connected with the bottom of the connector;
wherein when the connector is assembled into the front end of the barrel, the blocking members and the projections are guided to be blockably positioned through contact of the guiding faces and the inclines.

2. The syringe of claim 1, wherein the front end of the barrel further has a conical annular groove, and the conical annular groove and the engaging slot are configured to be stepped, and a diameter of the conical annular groove is greater than that of the engaging slot.

3. The syringe of claim 2, wherein the connector has a conical annular member arranged circumferentially, the conical annular member and the engaging member are configured to be stepped, the conical annular member is engaged correspondingly with the conical annular groove, the blocking member is engaged correspondingly with the engaging slot, an outer diameter of the conical annular member is greater than that of the blocking member, as the connector is installed in the barrel, the conical annular member is in close fit with the conical annular groove and the blocking member is blocked correspondingly within the engaging slot.

4. The syringe of claim 2, wherein the front end of the barrel further has a protrusion protruding from a lower edge of the conical annular groove, and the bottom of the connector is blockable by the protrusion.

5. The syringe of claim 1, wherein the interior of the connector integrally extends to form a restraining portion, the restraining portion extends inwardly and upwardly to form a tapered structure, and the bottom of the connector is formed with a recessed portion.

6. The syringe of claim 5, wherein a front end of the plunger is provided with a piston and a rod member which is protrusive outside the piston, the piston is movable and abuttable correspondingly against the bottom of the connector, the piston is fitted to the recessed portion, the rod member is received in the connector, and the rod member is fixedly attached to the tapered structure.

7. The syringe of claim 6, wherein the rod member is formed with a restraining slot, and the tapered structure is blocked within the restraining slot as the rod member is received in the connector.

8. The syringe of claim 1, wherein the connector has an extending member extending upwardly, and the extending member is for attachment of a needle assembly thereto.

9. The syringe of claim 8, wherein the front end of the interior of the barrel is formed with a threaded portion for threading connection of the needle assembly.

10. The syringe of claim 1, wherein each guiding face and each incline are V-shaped.
